(19) United States
(12) Patent Application Publication
(10) Pub. No.: US 2013/0028955 A1
(43) Pub. Date: Jan. 31, 2013

(54) SUSTAINED RELEASE ORAL MATRIX AND METHODS OF MAKING THEREOF

(71) Applicant: Gaurav Thakersi Tolia, Cincinnati, OH (US)

(72) Inventor: Gaurav Thakersi Tolia, Cincinnati, OH (US)

(21) Appl. No.: 13/190,398

(22) Filed: Jul. 25, 2011

(51) Int. Cl. A61K 9/20; A61K 31/277; A61K 31/196; A61K 31/573; A61K 47/34; A61K 47/32; A61K 47/12; A61K 47/10; A61K 47/26; A61P 29/00; A61P 9/06; A61P 31/04; A61P 31/12; A61P 7/02; A61P 3/10; A61P 25/08; A61P 35/00; A61P 31/10; A61P 25/06; A61P 33/02; A61P 3/04; A61K 31/167

(52) U.S. Cl. 424/450; 514/629; 514/523; 514/567; 514/180; 514/772.1; 514/772.6; 514/784; 514/772; 514/777; 424/465

(57) ABSTRACT

A solid dosage form suitable for forming a tablet for the containment and delivery of medicament is provided wherein the matrix forming material is a pressure sensitive adhesive, present in the amount from about 0.1 to about 40 weight %, based on the total weight of the composition. The dosage form is comprised of the medicament and a water-insoluble polymer silicone pressure sensitive adhesive and allows release of the medicament in a controlled fashion depending on simple parameters such as weight percent of the polymer silicone adhesive. A sustained release dosage form is provided for delivery of medicament wherein the release rate of medicament does not depend on the dissolution medium of the pH. Another aspect of invention is formation of solid tablets of poorly compressible material and the method for making the solid composition. The dosage form for this invention s particularly suitable for oral dosage forms.

### CROSS-REFERENCE TO RELATED APPLICATIONS

Non-provisional patent application filed using Attorney Docket No: 110-0290N 2010 Feb. 28 with the Title of the Invention: “pH-Independent Sustained Release Oral matrix and Methods of Making Thereof”

### STATEMENT REGARDING FEDERALLY SPONSORED RESEARCH OR DEVELOPMENT

Research partly funded by the NSF IGERT Program in Bio-Applications of Membrane Science and Technology (Grant #0333377) at the University of Cincinnati.

### THE NAMES OF THE PARTIES TO A JOINT RESEARCH AGREEMENT

The application for patent for the claimed invention discloses the names of Inventor Gaurav Tolia, an employer of the University of Cincinnati and the University of Cincinnati where the claimed invention was undertaken as the parties to the joint research agreement.

### INCORPORATION-BY-REFERENCE OF MATERIAL SUBMITTED ON A COMPACT DISC

Not Applicable

### BACKGROUND OF THE INVENTION

(1) Field of the Invention

The present invention relates generally to the use of pressure sensitive PSA for the formation of oral solid matrix tablet of medicament(s) not requiring any additional excipients for aiding compression and release, and particularly, to a sustained release dosage form matrix of active agent(s) providing drug release for an extended period of time after administration.

(2) Background of the Invention

Generally oral administration of medicaments remains the most convenient and preferred route. A variety of substances, such as pharmaceutical active drugs and nutraceuticals are often formulated in a solid dosage form such as tablets, coated tablets, pastilles, pellets, granules and the like. It is herein referred as the term “solid dosage form” to a presentation, which is suitable in particular for oral, buccal or rectal administration in any of desired forms and shapes. The term “tablet” is a term well understood by not only those skilled in the art but also to the general public at large. Generally, the term “tablet” includes not only tablets which are oval or cylindrical in shape per se but also includes similar discrete bodies, perhaps of other shapes such as donut for example and sometimes known by different names, such as “caplets” (e.g. capsule-shaped tablets), lozenges, and pills. Herein it is referred as a mixture of medicament with the pressure sensitive PSA and other excipients if required which have been brought together by various ways and compressed using one or more apparatus known to those skilled in the art so that the medicament(s), the polymer and excipient(s) becomes a compacted “tablet” which is free of chipping, cracking or lamination and the “tablet” is capable to persist normally encountered handling conditions. This compacted “tablet” is able to resist disintegration at the desired time, site, or combination of both.

Generally, tablets contain a medicament and at least an excipient such as lubricant, granulating agent, binders, antiadherents, glidants or combinations thereof in order to form a tablet of poorly compressible powder for the formation of a tablet which is free from defects such as chipping, cracking or lamination for example. The use of “tableting aid” are generally known to those skilled in the art of making tablets. Additionally, the process of making tablets using direct compression involves process of granulation (wet or dry) of poorly compressible material. The processes used for making direct compression tablets of poorly compressible medicaments is also known to those skilled in the art of making tablets.

In a pharmaceutical context, a poorly compressible medicaments are usually powders, which undergo elastic deformation when compressed under high pressure using any equipment's used for making solid dosage forms. During tablet manufacturing, solid powders are compressed under high pressure in a punch and a die. Poorly compressible powders deform under high pressure in a punch and a die during tablet compression. On removal of the pressure during ejection of the tablet from the die, the elastically deformed powders undergo elastic recovery and expand. This is supposed to be one of the causes leading to chipping, cracking or lamination of the tablet on ejection from the tablet press equipment. Those skilled in the art solve this problem by either adding excipients such as binders, lubricants, glidants or disintergrants or using processes which are known to those skilled in the art. In addition, those skilled in the art also solve the problem by using method to form granules of poorly compressible powder. U.S. Pat. Nos. 5,370,878 and 5,733,578 discloses directly compressible compositions of poorly compressible drug acetaminophen. The disclosures use more than one excipient for development of compressible acetaminophen. The process used is wet granulation, which requires more than two steps to produce compressible compacts, and is time consuming.

There is much art and skill in practicing the process of wet granulation, which involves addition of medicament powder with granulating fluid and forming a mix, which is then dried to obtain granules. It is generally recognized that tableting aids are required to form moist granules of fairly uniform size. The granules are then forced through a mesh screen to maintain homogeneity of mass. The process also requires drying the moist, screened mash and it is important that the mass does not agglomerate. binders, glidants, lubricants and disintegrants. The terms of tableting aids such as “binders” means to one skilled in the art a substance, which helps to bind the particles of powder together in a form suited to compaction and compression. A “glidant” means a substance which aid the medicament granules to fill into the die before compression. A “lubricant” means a substance, which helps the compressed tablets to leave the die and not stick to the walls of the die. A “disintegrant” means a substance, which helps the tablet to disintegrate in desired, site or time.

One problem that is known with use of tableting excipients such as granulating agents, binders, lubricants or glidants do not necessarily sustain the release of the medicament. Sustained release oral dosage form enhances the pharmacological benefits for numerous medicaments. These sustained release dosage forms reduce the frequency of administration of the dosage form. In order to sustain medicament release from the tablet, the medicament is coated using controlled release polymers known to those skilled in the art. It is well know that coating of drug particles requires expensive equipment's and requires many unit processes.

There is much art and skill in practicing the use of polymeric materials such as celluloses, gums, and acrylates for example for sustaining release rate of medicament. U.S. Pat. Nos. 5,733,031 and 5,858,412 describes compositions and methods for producing directly compressible tablets for sustained release of medicaments. None of the disclosures describe use of silicone PSA or a pressure sensitive PSA for development of solid tablet of poorly compressible drugs. It involves either coating the drug powder with controlling polymer. The equipment's used for coating drug powder are very expensive and are time consuming. Alternatively, sustained release tablets are prepared by mixing of powder of polymeric materials with medicament powder and compressible into a tablet. Simple mixing of the polymer with poorly compressible powder does not solve the commonly known problem of capping, chipping or lamination of the compressed “tablets”. Therefore, use of polymers with known art and skill for practice of sustained release oral tablets does not solve the problem of formation of tablets with sufficient mechanical strength, which can resist normally encountered handling conditions. which can resist of poorly compressible drugs without use of excipients such as glidants, lubricants, granulating agents or binders. Therefore, oral tablets, which are designed to sustain release of medicaments either, use coating, or polymers require additional tableting excipients. There are no simple techniques for making tablets of poorly compressible drugs without wet granulation or requiring tableting excipients such as binders, glidants, lubricants or granulating agents.

Therefore, there is a need for a polymer which can provide controlled release oral delivery system where the drug release can be controlled by inclusion of small amount of polymer which allows formation of tablets with sufficient mechanical strength of poorly compressible medicament. There is also an advantage if the polymer used for controlling drug release and aiding compression does not interact with the gastric environment and hence provide steady reliable release rate of medicaments irrespective of the pH of the gastric environment or the dissolution conditions.

Surprisingly, it now has been found that employing polymer such as silicone pressure sensitive PSA provides compositions for oral tablets containing poorly compressible drug acetaminophen. The tablets produced showed sufficient mechanical strength and can provide sustained release of incorporated medicament without addition of known tableting excipients or requiring expensive coating equipment's. Surprisingly, it is also found that addition of silicone pressure sensitive PSA in oral tablet composition allows release of medicament to remain unchanged irrespective of the pH of the dissolution medium, ionic strength of the dissolution medium or the presence of mechanical agitation of the dissolution medium.

Use of pressure sensitive PSA for drug delivery application to skin or devices is well known in the art. Generally PSA's are used for adhering to a substrate or adhering two substrates. The use of PSA's for adhesion is described for example in U.S. Pat. Nos. 2,857,356, 5,234,957 and 3,797,494. “PSA”'s are generally defined as materials which forms bond with the substrate when light pressure is applied. It is well known to those in the art that PSA's generally consists of an elastomer linked with suitable resins. The elastomer is well known to be based on either acrylic, butyl ether, vinyl ether, ethylene vinyl acetate, natural rubber, nitriles, styrene block copolymers or silicone rubbers for example. The process for mixing the PSA with the medicament to form an adhesive device capable of adhesion is also generally well known in the art. U.S. Pat. Nos. 2007/0,166,244 and 2006/0,142,411 discloses invention related to use of silicone pressure sensitive adhesive for delivery of tooth whitening agents or oral care substance to the oral cavity. None of the disclosures relate to use of unique properties of pressure sensitive adhesive for improving compressibility of poorly compressible medicaments for oral sustained drug delivery. Moreover, the disclosure do not relate to sustained release of medicament along the gastro intestinal tract for oral drug delivery. Herein, “oral drug delivery” refers to delivery of drug along the gastrointestinal tract (GI). This is although not limited to the oral GI system but it is also applicable to rectal, buccal and oral cavity.

Surprisingly, it is now found that addition of solid medicament or excipients in amount greater than 60 weight percent in adhesive reduces the adhesive properties of the PSA's and the formed solid matrix is compressible. This compressible solid matrix of small amount of PSA(s) and medicament(s) without any tableting excipient can be compressed under high pressure to produce tablets of sufficient mechanical strengths.

### SUMMARY OF THE INVENTION

The present invention is a solid composition capable of forming tablets of sufficient mechanical strengths. The solid composition comprises of pressure sensitive adhesive, in an amount sufficient for formation of tablets with good mechanical strengths when compacted: from 0.1 to about 40 weight % based on total weight of the composition, wherein the medicament is the remaining substance in the composition other then the PSA. Generally, the PSA of the present invention includes a silicone tackyfying resin and polyorganosiloxane

Another aspect of the present invention is the process for preparing a solid composition and particularly one suitable for tableting comprising of dissolving the PSA in a solvent in a predetermined amount; addition of medicament powder at room temperature; mixing to form a suspension of medicament in PSA solution; drying of the suspension; compression of the dried material to form a substantially homogenous matrix tablet free of defects such as chips, cracks or lamination.

It is an object of the present invention to provide a solid composition that is suitable for manufacture of tablets capable of providing sustained release of medicament wherein the release rate does not depend on the pH of the dissolution medium or presence of mechanical agitation.

According to yet another aspect of the invention, the dosage form does not depend on presence of buffers or tableting excipients for the formation of tablets with sufficient mechanical strength or to provide sustained release of medicaments.

### BRIEF DESCRIPTION OF THE SEVERAL VIEWS OF THE DRAWING(S)

FIG. 1 A) is an illustration of stereomicroscopic images of the tablets showing chipping on edges when prepared using currently used polymer, ethyl cellulose and poorly compressible drug acetaminophen, and B) is an illustration of stereomicroscopic images of the tablet free of chipping and cracking prepared using polymer in this invention, a silicone pressure sensitive adhesive (PSA) and poorly compressible drug acetaminophen produced according to Example 1F and Example 2 respectively.

FIG. 2 shows mean of cumulative drug release after dissolution testing in different pH medium from silicone PSA tablets of different weight percent according to Examples 1F.

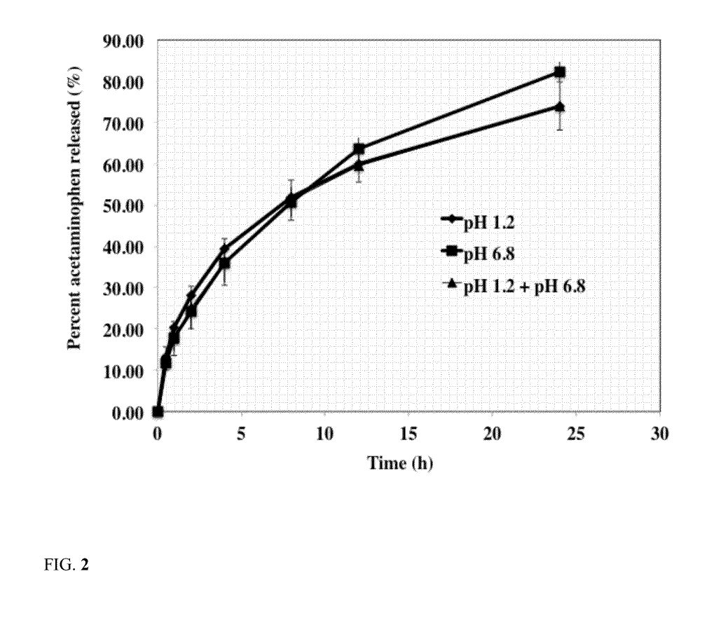

FIG. 3 shows mean verapamil hydrochloride drug release after dissolution testing from silicone PSA tablets containing 20 weight percent silicone PSA in different dissolution medium pH according to Example 3.

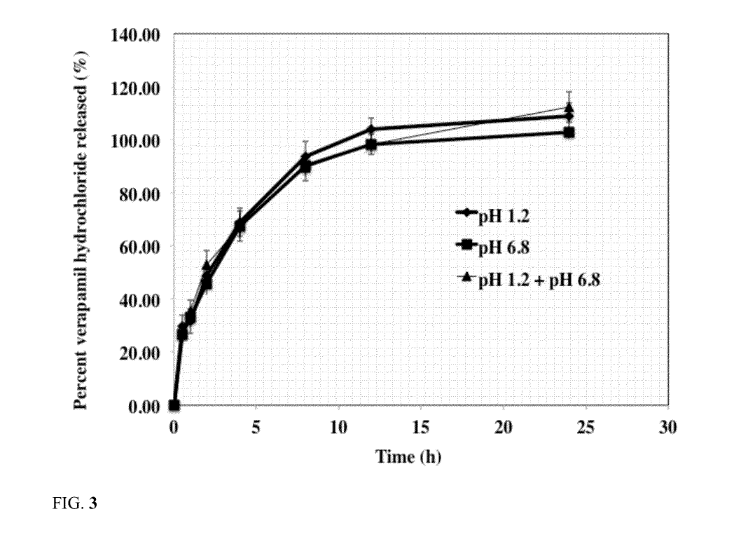

### DETAILED DESCRIPTION OF THE INVENTION

The present invention is based on the unusual discovery that solid matrix containing using silicone pressure sensitive adhesive (PSA) are useful for formation of tablets under high pressure with sufficient mechanical strengths of poorly compressible medicaments. Herein is provided a solid oral matrix tablet specifically containing at least above 0.1 weight percent of silicone PSA and below 99.9 weight percent of one or more of pharmaceutical, nutraceutical, veterinarian, cosmaceutical or dietary supplement active medicament(s) and mixtures thereof. It is understood that while the embodiments described refer to the use of silicone PSAs and silicone PSA's in particular, other adhesives and PSA's which are viscoelastic, inert, water-insoluble and capable of being compressed under slight pressure can also be useful for formation of solid oral tablets with sufficient mechanical strengths and in providing sustained release of medicaments.

There is provided a novel use of pressure sensitive adhesive materials for improving compressibility of poorly compressible materials. Pressure sensitive adhesives can deform under slight pressure, while elastic drug powders undergo elastic recovery when compressed under pressure. Use of mixture(s) of deformable polymers with elastic powders for direct compression enables improvements in the properties of the formed tablet is described in this invention. The novel use of these pressure sensitive adhesive materials provides directly compressed tablets with excellent mechanical properties of medicaments, which are poorly compressible. The material is also hydrophobic, inert, water insoluble with low surface energy and glass transition temperature. Hydrophobic and low surface energy properties of the material are utilized for novel application of coating the drug powder for oral sustained release drug delivery by mixing the material directly with the active medicaments.

Use of inherent properties of pressure sensitive adhesives is utilized for a novel use, which allows improvement in drug release characteristics suitable for oral drug delivery application. The physiological conditions along the gastrointestinal tract changes with respect to pH, ionic strength, and the rate and extent of gastric motility. It is well know in to those skilled in the art that oral controlled release drug delivery systems require polymers to provide robust release rate. The polymers used currently for oral controlled release are in glassy state. These conventionally used polymer for commercial products of oral controlled release include polymers that have glass transition temperature above room temperature and are non-cohesive in nature. Hydrophilic polymers known to those skilled in the art are polymers, which swell, dissolve, erode or combinations thereof and thereby provide controlled release rate after interacting with water environment. The disadvantages of hydrophilic polymers include uptake of large amount of water when exposed to aqueous environment of the gastrointestinal tract. They also change their state from glassy state to rubbery state when hydrated. In doing so, they depend on the aqueous environment and hence are prone to show effect of dissolution medium pH on drug release rate. There is provided an improvement over hydrophilic polymers for controlled oral release tablet using silicone pressure sensitive adhesive where the release rate of drug from silicone PSA tablets do not depend on the pH of the dissolution medium.

Use of the silicone pressure sensitive adhesives [PSA] is the material described here for improvement in oral controlled release tablets has low glass transition and are highly cohesive material. Formation of matrix with the material provides improvement because the cohesive nature of the material allows the prepared tablet to sustain mechanical agitation of the gastrointestinal physiology in form of peristalsis and gastric motility and remain cohesive and not fall apart and loose its controlled release mechanism. Non-cohesive polymers that are currently used for controlled release and widely known to those skilled in the art have high glass transition temperature (Tg). It has been illustrated that matrix tablet prepared using non-cohesive high Tg polymers are unable to sustain their drug release mechanism on presence of mechanical agitation.

Silicone PSA is an exemplary matrix with necessary characteristics because it has been utilized in commercial transdermal, topical, wound care and medical devices and has been tested for its biocompatibility and toxicology. Silicone PSAs have also been used for transmucosal and transbuccal and for oral applications. The inertness of silicones is useful and hence it is used in over 50% of the cosmetic products as well as long for long term implant applications. Silicone PSA is resistant to water and does not absorb water. Normally encountered physiological medium does not break down silicone PSA and therefore can be considered as inert polymer. Due to inert and hydrophobic nature of silicone PSA, the oral matrix tablet formed using silicone PSA is able to provide sustained release of contained medicament(s) at same rate irrespective of the pH of the dissolution medium (between pH 1.2 and 6.8).

Although silicone materials such elastomers, silicone liquid, silicone adhesive has been known to provide sustained release of active medicaments, pressure sensitive adhesives differ in their properties which has not been utilized for direct compression of poorly compressible drugs such as acetaminophen for example. Use of silicone elastomers for sustained release is well now to those skilled in the art. Use of pressure sensitive adhesives for helping compression of poorly compressible drug powder is not obvious. Adhesives can stick to substrates they come in contact with and hence would not be obvious choice for use as a material for oral direct compression process.

The process described here is an improvement in utilizing the property of pressure sensitive adhesives for direct compression by addition of solid drug powder in high loading with an adhesive. By addition of high drug loaded powder which does not dissolve in the solvent during mixing of adhesive and the drug powder, the adhesion surface of the adhesive is taken by the drug powder. The overall adhesion of the pressure sensitive adhesive (PSA) is greatly reduced and the solid matrix does not stick to the die and punches during compression. Silicone PSA as used in transdermal drug delivery devices. Transdermal devices are prepared by dissolving the drug in the solvent along with the adhesive. The formed device is required to be sticky and adhere. The novelty in this invention utilizes the fact that a reduction in adhesion of PSAs can be achieved by suspending high drug loading powder in the adhesive matrix. This process and the composition reduces the disadvantages of adhesion properties of the adhesive. The improvement then is shown where the compressibility properties of adhesives are utilized for forming solid tablets.

Acetaminophen commonly known as paracetamol, is a widely used analgesic and antipyretic drug, which is widely known to those skilled in the art because of its poor flowability and compressibility properties. It is often used alone or as combination in commercially available products with drugs such as tramadol hydrochloride, caffeine, pseudoephedrine, diphenhydramine, pamabrom and butalbital. The commercially available form of acetaminophen is the monoclinic powder form. This powder form of acetaminophen is well known to undergoes particle fragmentation and elastic deformation, which leads to capping and chipping of formed tablets during tablet compression. Wet granulation is a routinely employed procedure for improving compressibility of poorly compressible drugs such as acetaminophen and it is well know to those skilled in the art.

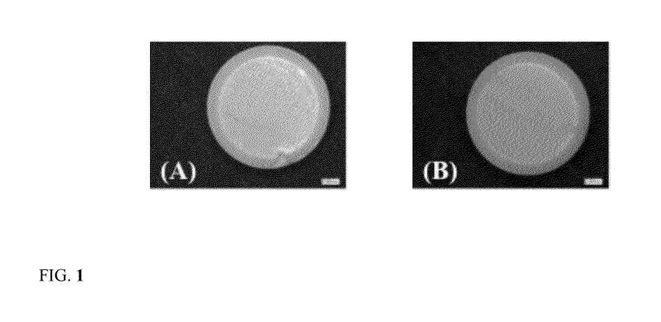

As illustrated in FIG. 1. tablets prepared using silicone PSA do not show the problem of chipping, cracking or lamination. In comparison, tablets prepared using rigid polymer ethyl cellulose how chipping and cracking Also, the friability of silicone PSA tablet is very low. The mechanical strength of silicone PSA tablet is illustrated in example II. It has been illustrated that the mechanical strength of these tablets formed using silicone PSA containing poorly compressible drug acetaminophen is surprisingly high. In comparison, mechanical strength of tablet containing ethyl cellulose is low and shows high friability.

As illustrated in Example II, the contact angle of silicone PSA tablet shows high value. This is improvement compared to use of water insoluble hydrophobic polymer such as ethyl cellulose. High hydrophobic surface provided by silicone PSA in this invention is useful for sustaining release of water soluble drug acetaminophen using as little as 5 weight percent of silicone PSA for 12 h. This indicates that silicone PSA can serve as a matrix for development of sustained release tablets and it can eliminate the compressibility issues normally encountered when compressing poorly compressible powders.

The process disclosed here is novel and simpler compared to that known to those skilled in the art of coating. The process of coating drug powder requires expensive equipment's and the coated dug particles are required to be sieved after drying. In case of silicone PSA, the particles are ready to be compressed and when compressed, they show uniformity due to excellent spread ability of silicone PSA over substrates.

Example IV shows an improvement in controlling drug release rate by using silicone PSA due to the effect of dissolution medium pH. Example VI shows an improvement for oral delivery for release of medicament using silicone PSA when exposed to bio relevant dissolution medium. Bio relevant dissolution medium as tested in Example VI contain bile salts and lecithin in order to mimic fed and fasted state of the aqueous environment in vivo. The silicone PSA tablets tested under dissolution conditions shows that there is no change in release rate of acetaminophen. Ethanol is sometimes consumed by patients when a controlled release tablets is to be required to be administered. Ethanol consumption has shown to disrupt the mechanism of controlled release dosage form leading to dose dumping and causing unwanted side effects of therapeutic inefficacy. Silicone PSA tablets show that presence of ethanol does not affect the drug release rate and drug release mechanism and does not lead to dose dumping.

As illustrated in Example X, silicone PSA tablets when exposed to mechanical stress do not show change in the drug release rate. In comparison, conventional polymers, which are rigid and glassy in state, do not sustain mechanical stress application. Application of stress on ethyl cellulose tablets show burst release indicating loss of controlled release mechanism. Silicone PSA is a cohesive polymer and its use for oral delivery system where mechanical stress is normally encountered due to gastric motility and peristalsis. Use of cohesive polymer such as silicone PSA is shown here as an improvement over conventional polymers known to those skilled in the art for oral drug delivery.

The use of the improved material will reduce formulation development time and effort to obtain appropriate composition for eliminating chipping and cracking issues related to direct compression of poorly compressible medicaments which are required in high dose. The use of the improved material eliminates the need for use of binders, lubricants, glidants or granulating's agents. The use of the improved material also eliminates the need for use of expensive and time consuming process of drug coating. Low surface energy of the material ensures coating of drug powder and the hydrophobic property of the silicone coating formed prevents water from entering the matrix and hence provides sustained release of medicament.

Therefore, the use of pressure sensitive adhesives and the process disclosed herein reduce an improved material exhibiting unexpected improvement in compressibility of poorly compressible drug powder at high loading and also provide sustained release of medicament which do not change depending on the outside dissolution medium pH conditions.

### Example I

### Images of Tablets Formed Using Conventional Glassy Polymer in Comparison to Silicone PSA Polymer Tablet

The present composition is prepared using conventional polymer, ethyl cellulose that is an example of a glassy polymer with high glass transition temperature. Ethyl cellulose is an example of a polymer, which is widely used in oral controlled release tablet formulations for development of sustained release products. Tablets were prepared for this example by dissolving about 2 g of ethyl cellulose in about 7 ml of ethyl acetate. Acetaminophen powder was sieved and about 8 g of acetaminophen powder was added to the vial containing ethyl cellulose or silicone PSA solution. The vial was allowed to rotate for 4 h and the suspension obtained was poured onto a fluoropolymer coated side of the release liner. The suspension was allowed to dry overnight. Weighed 500 mg portions of the dried matrix was compressed using a 13 mm flat-faced cylindrical die in a Carver press at 6.4 kN force for 2 sec. The tablets obtained are illustrated in FIG. 1. The composition of the tablets is 20 weight percent of polymer and 80 weight percent of acetaminophen. After the tablet preparation, the obtained tablets were imaged using a stereomicroscopic to investigate the effect of addition of glassy polymer for formation of tablet of poorly compressible drug acetaminophen. From FIG. 1, it can be seen that the tablets obtained using glassy polymer such as ethyl cellulose does not produce tablet with good mechanical strength. There is chipping of the tablet, which is not acceptable for a good product.

The problem of chipping, cracking or lamination of tablets prepared using ethyl cellulose is not exclusive. This problem has been shown in examples for other currently used polymers and excipients such as manifold, celluloses, or acrylates for example. There is no benefit obtained by using glassy polymers such as ethyl cellulose for development of directly compressible tablet of poorly compressible drug such as acetaminophen suing simple procedure as described in this example.

### Example II

### Mechanical Strength Evaluation of Silicone PSA Tablets

The mechanical properties of the formed tablet containing various weight percent of silicone PSA and acetaminophen from Example I may be evaluated using the procedure such as tablet hardness and tablet friability. The hydrophobicity of tablet surface may be evaluated using the technique of contact angle measurement. Higher contact angle values indicate hydrophobic surface. In order to measure tablet hardness, a tablet is placed between two holders. The holders move to apply force on the tablet. The force required to break a tablet is presented as tablet hardness value. An average of three measurements are shown in Table 2. Tablet friability is measured by placing three tablets in a revolving friability tester. In order to test friability of tablet composition, three tablets were revolved for 100 revolutions. The weights of the tablets before and after friability testing were obtained. % Friability value may be calculated from the total tablet weight of intact tablets before and after friability testing. Contact angle measurements were performed using a Rame-Hart Goniometer using sessile-drop method. A 5 ul drop of water is placed on the surface of a tablet and the angle of water droplet formed on the tablet surface is measured.

Tablet hardness of various tablets containing silicone PSA is shown in Table 2. The tablet hardness increases as the weight percent of silicone PSA in the tablet composition increases. Silicone PSA is acting as a binder of poorly compressible drug acetaminophen (APAP). Friability of tablet was measured using friability tester and the results are shown from example 1A-F. Increasing the silicone PSA composition above 5 weight percent shows acceptable friability. A pharmaceutically acceptable value of friability of a tablet is a value less than 1%. The friability of silicone PSA tablet containing 20% weight polymer was compared with standard conventional polymer ethyl cellulose based tablet. The friability value of conventionally used polymer tablet of ethyl cellulose was 4.3%, which is higher than pharmaceutically acceptable value for a oral tablet. The contact angle of all the silicone PSA tablets shown in Examples 1A-1F show high values. In contrast, contact angle value on ethyl cellulose tablet shows a low value of 44.1 degrees. Low contact angle value on ethyl cellulose tablet indicate the difference between using a hydrophobic polymer with high and low glass transition temperature and viscoelasticity and spreading ability of the polymer film on solid drug powder surface. Silicone PSA has low surface energy of 23 dynes/cm², and hence it is capable of spreading better as compared to rigid polymer such as ethyl cellulose. These examples also signifies the novelty in this invention of using polymer with low surface energy and low glass transition temperature and its ability to provide a hydrophobic surface for sustained release of water soluble drug in addition to its ability to aid compression of poorly compressible powders. The glass transition temperature of silicone PSA is −123 C.

### Example III

### Release of Acetaminophen from Silicone PSA Tablets

The effect of silicone PSA weight percent in tablet composition on release rate of acetaminophen from the tablets was studied using the following procedure. The results of acetaminophen release from silicone PSA tablets are shown in Table 3.

The release rate of these formulations were carried out using USP dissolution basket apparatus 1 (Vankel, Inc) using about 100 rpm stirring speed at about 37 C in about 900 ml of the dissolution medium. The dissolution medium used was simulated intestinal fluid with a pH of about 6.8. At predetermined time intervals, 1 ml of sample was withdrawn and was analyzed to determine acetaminophen concentration. Concentration of acetaminophen in the dissolution medium was determined using a HPLC method. A C8 column was used using mobile phase of 65% methanol and 35% water. Flow rate was 1 ml/min and the UV detection wavelength was 254 nm. The results are reported as average of six tablets. Results indicate that increase in silicone PSA composition in the matrix decreases the release rate of acetaminophen from the tablet matrix. Total weight of each tablet was 500 mg. The diameters of the tablets were 13 mm.

It can be seen that increasing the amount of silicone PSA in the composition decreases the release rate of acetaminophen from the matrix. The results indicate the importance of silicone PSA in controlling the release rate of active medicament, acetaminophen from the silicone PSA tablet. Small amount of 5 weight percent of silicone PSA is also capable of sustaining release of acetaminophen for 8 h.

### Example IV

### Release of Acetaminophen from Silicone PSA Tablets in Different Dissolution Medium pH

The effect of dissolution medium pH on drug release from silicone PSA tablet was determined using silicone PSA composition from example 1F. The silicone PSA tablet tested for pH effect contained 20 weight percent of silicone PSA BIO PSA 7-4202 and 80 weight percent of acetaminophen.

The conditions for the dissolution study in this example is similar to that explained in Example III, except one study was performed using dissolution medium of simulated gastric fluid at about pH 1.2. The rate of acetaminophen release from 20 weight percent silicone PSA tablet in either about pH 1.2 or about pH 6.8 dissolution medium was evaluated and compared. The result shown in Table 4 indicates that the release rate of acetaminophen from silicone PSA tablet does not change due to the dissolution medium pH.

### Example V

### Release of Acetaminophen from Silicone PSA Tablets Compressed Using Different Compression force

The effect of compression force on the release rate of acetaminophen from silicone PSA tablets obtained was evaluated using dissolution medium pH simulated intestinal fluid at about pH 6.8. The compression force used for making silicone PSA tablet is shown in Table 5 along with the corresponding release rate of acetaminophen from the silicone PSA tablets. The silicone PSA tablet tested for pH effect contained 20 weight percent of silicone PSA BIO PSA 7-4202 and 80 weight percent of acetaminophen.

| | Example | | |
|---|---|---|---|
| | VA | VB | VC |
| Polymer | Silicone | Silicone | Silicone |
| % w/w polymer | 20 | 20 | 20 |
| % w/w APAP | 80 | 80 | 80 |
| Compression force (kN) | 3.5 | 6.1 | 8.7 |

Silicone here is Bio-PSA 7-4202 amine compatible adhesive resin from Dow Corning

The conditions for the dissolution study in this example is similar to that explained in Example III. The rate of acetaminophen release from 20 weight percent silicone PSA tablet compressed at three different compression force was evaluated in about pH 6.8 dissolution medium and compared. The results are shown in Table 5. The results indicate that the compression force does not affect the release rate of acetaminophen from silicone PSA tablets.

### Example VI

### Release of Acetaminophen from Silicone PSA Tablets in Bio Relevant Dissolution Medium

The effect of dissolution medium composition on drug release from silicone PSA tablet was determined using silicone PSA composition from example 1F. The silicone PSA tablet tested for pH effect contained 20 weight percent of silicone PSA BIO PSA 7-4202 and 80 weight percent of acetaminophen compressed using 6.1 kN compression force.

The conditions for the dissolution study in this example is similar to that explained in Example III, except the composition of the dissolution medium. Bio relevant dissolution medium are commonly used to evaluate the effect of presence of bile salts on drug release. Fed and fasted state bio relevant dissolution medium are especially relevant for studying the inertness of controlled release polymer. The rate of acetaminophen release from 20 weight percent silicone PSA tablet in either 10% ethanol solution, fed or fasted state bio relevant dissolution medium was evaluated and compared. The results are shown in Table 6. The results indicate that the release rate of acetaminophen from silicone PSA tablet does not significantly change due to the dissolution medium composition.

### Example VII

### Release of Acetaminophen and Tramadol Hydrochloride from Silicone PSA Tablets

The effect of addition of another drug in the solid tablet matrix may be evaluated by changing the composition of the silicone PSA tablet. The compositions in Example VII are shown in table below. Tramadol hydrochloride was added using the procedure as described in example III. The release rate of acetaminophen and tramadol hydrochloride was evaluated in this example in simulated intestinal dissolution medium at about pH 6.8. The dissolution testing conditions are similar to that described in Example III. The silicone PSA tablet tested contained 20 weight percent of silicone PSA BIO PSA 7-4202, about 8.7 weight percent tramadol hydrochloride and about 71.7 weight percent of acetaminophen compressed using 6.1 kN compression force. The results are shown in Table 7 below. The concentration of tramadol hydrochloride and acetaminophen from the dissolution samples were determined using a HPLC method. A C18 HPLC column was used to quantitate both drugs in the dissolution samples at 270 nm, using about 40 volume percent acetonitrile in 60 volume percent of 0.05% w/v trifluoroacetic acid aqueous mobile phase. The result for release rate for tramadol hydrochloride and acetaminophen indicates that silicone PSA is able to sustain release rate of two drugs when mixed in same tablet. Also, the release rate of both drugs tramadol hydrochloride and acetaminophen remain unchanged from the silicone PSA tablet described in this invention. This shows an improvement in oral matrix, where the release rate of a drug is unchanged on addition of another drug by changing the composition of the tablet. The improvement in this said invention will allow those skilled in the art to decrease in time to optimize the formulation and decreases the variable involved in controlling drug release rate.

### Example VIII

### Release of Acetaminophen from Silicone PSA Tablets Containing Surfactants

Silicone PSA tablet compositions according to the present invention are shown below in the amounts of components in weight percent. The compositions are made using procedure described in Example VIII. The tablets were prepared using the process described in Example III.

| Components | VIIIA | VIIIB | VIIIC | VIIID |
|---|---|---|---|---|
| Silicone PSA | 10 | 10 | 10 | 10 |
| Acetaminophen | 80 | 80 | 80 | 80 |
| Capmul 50 | 10 | — | — | — |
| Tefose1500 | — | 10 | — | — |
| Brij98 | — | — | 10 | — |
| Myrj53′ | — | — | — | 10 |

Silicone here is Bio-PSA 7-4202 amine compatible PSA resin from Dow Corning

Capmul 50 here is glyceryl monoleate obtained from Abitec Corporation
Tefose1500 here is PEG stearate from Gattefosse
Brij 98 here is Polyoxyethylene (20) Oleyl Ether from Sigma Aldrich
Myrj53 here is Polyethylene 50 stearate from Croda

Release of acetaminophen from silicone tablets described in VIIIA-D is shown below in Table 8. The procedure for determining acetaminophen drug release rate from these tablets was described in Example III. Addition of surfactants in silicone matrix changes the release rate of acetaminophen in comparison to the silicone tablet without surfactant being present in the composition. The surfactants shown herein are examples. Change in release rate may be possible with other excipients, which act as surface active agents. Addition of surfactants to silicone PSA matrix changes the release rate depending on the type of surfactant used.

### Example IX

### Release of Acetaminophen from Silicone PSA Tablets Containing Excipients

Silicone PSA tablet compositions according to the present invention are shown below in the amounts of components in weight percent. The compositions are made using procedure described in Example IX A-D. The tablets were prepared using the process described in Example III.

| Components | IXA | IXB | IXC | IXD | IXD |
|---|---|---|---|---|---|
| Silicone PSA | 20 | 20 | 20 | 10 | 10 |
| Acetaminophen | 80 | 60 | 60 | 90 | 80 |
| Mannitol | — | 20 | 20 | — | — |
| PVP K90 | — | — | 20 | — | — |
| Tris | — | — | — | — | 10 |

Silicone here is Bio-PSA 7-4202 amine compatible adhesive resin from Dow Corning

Release of acetaminophen from silicone tablets described in 1×A-D is shown in Table 9. Addition of excipients in the silicone matrix does not change the release rate of acetaminophen in comparison to the silicone tablet without excipients being present. The excipients shown herein are examples. Commonly known excipients to those in the art may also be substituted in the silicone matrix to obtain sustained release tablet containing medicament.

### Example X

### Effect of Mechanical Stress on Release from Silicone Tablet

The effect of mechanical agitation on drug release from silicone PSA tablet was determined using silicone PSA composition from example 1F. The silicone PSA tablets tested for pH effect contained about 20 weight percent of silicone PSA and about 80 weight percent of acetaminophen compressed using 6.1 kN compression force. The results were compared to tablets prepared using 20 weight percent ethyl cellulose and 80 weight percent acetaminophen. Three different silicone PSAs with varying resin/polydimethyl siloxane ratio were prepared using procedure described in example III and compared. The three different silicone PSAs BioPSA 7-4102, 7-4202 and 7-4302 vary in their extent of adhesion to a substrate.

The conditions for the dissolution study in this example is similar to that explained in Example III for (−) without mechanical agitation. Effect of mechanical agitation of performed using procedure described by Garbacz and Weitschies. Program 3 of the novel stress test apparatus was used to evaluate the effect of mechanical stress on drug release. Pressure waves and 100 rpm rotation were used to induce stress on tablets at predetermined time points and drug release determined. The results for acetaminophen drug release using USP apparatus 1 (−) and in stress test apparatus (+) is shown in Table 10. The results indicate that silicone PSA tablets do not show effect of mechanical stress on drug release. In comparison, ethyl cellulose tablets show burst release when subjected to mechanical stress in comparison to tablets without being subjected to mechanical stress. This effect of preventing burst release on application of mechanical stress is an improvement from current literature. Silicone adhesive matrix tablets are shown to provide robust mechanism of controlling drug release.

### Example XI

### Release of Verapamil Hydrochloride from Silicone PSA Tablets

The effect of dissolution medium pH on drug release from silicone PSA tablet containing weakly basic drug verapamil hydrochloride was determined using USP apparatus 1 dissolution apparatus at about pH 1.2 and about pH 6.8. Verapamil hydrochloride was present in 80 weight percent and silicone PSA was present in 20 weight percent. Tablets were prepared using procedure described in example III. The silicone PSA tablet tested for pH effect contained 20 weight percent of silicone PSA BIO PSA 7-4202 and 80 weight percent of verapamil hydrochloride. The effect of dissolution medium pH on drug release from ethyl cellulose tablet was tested for comparison.

The conditions for the dissolution study in this example is similar to that explained in Example III, except one study was performed using dissolution medium of simulated gastric fluid at about pH 1.2 and one in simulated intestinal fluid about pH 6.8. The rate of verapamil hydrochloride release from 20 weight percent silicone PSA or ethyl cellulose tablets in either about pH 1.2 or about pH 6.8 dissolution medium was evaluated and compared. The results are shown in Table 11. The results indicate that the release of verapamil hydrochloride from silicone PSA tablets is not affected by the change in the dissolution medium pH. In comparison, glassy rigid polymer tablet prepared using ethyl cellulose shows that verapamil hydrochloride release is dependent on the pH of the dissolution medium.

### Example XII

### Release of Different Medicaments from Silicone PSA Tablets

Silicone PSA tablet compositions according to the present invention are shown below in the amounts of components in weight percent. The compositions are made using procedure described in Example XII.

| Components | XIIA | XIIB | XIIC | XIID |
|---|---|---|---|---|
| Silicone PSA | 20 | 20 | 20 | 20 |
| Diclofenac sodium | 80 | — | — | — |
| Sodium salicylate | — | 80 | — | — |
| BSA | — | — | 80 | — |
| Tramadol HCl | — | — | — | 80 |

Silicone here is Bio-PSA 7-4202 amine compatible adhesive resin from Dow Corning

Release of various drugs from silicone tablets described in XIIA-D are shown below in Table 12. It is possible to prepare tablets containing medicaments with different physicochemical properties using silicone PSA. Sustained release of various medicaments is shown from results obtained in Table 12 below. The dissolution study was described in detail in example III.

### Example XIII

### Release of Verapamil Hydrochloride from Acrylate Pressure Sensitive PSA Tablets

Tablet compositions according to the example XIII are shown in tablet below. The procedure used to prepare this composition is described in detail in Example III. The tablets were 500 mg total weight and similar dimensions as described in example III. The effect of dissolution medium pH on drug release from acrylate PSA tablet containing weakly basic drug verapamil hydrochloride was determined using USP apparatus 1 dissolution apparatus at about pH 1.2 and about pH 6.8. Verapamil hydrochloride was present in 80 weight percent and acrylate PSA was present in 20 weight percent. Tablets were prepared using procedure described in example III. The acrylate PSA tablet was tested for pH effect. The acrylate tablet composition was about 20 weight percent of DURO_TAK 87-202A acrylate PSA and about 80 weight percent of verapamil hydrochloride.

The conditions for the dissolution study in this example is similar to that explained in Example III, except one study was performed using dissolution medium of simulated gastric fluid at about pH 1.2 and one in simulated intestinal fluid about pH 6.8. The rate of verapamil hydrochloride release from 20 weight percent acrylate PSA in either about pH 1.2 or about pH 6.8 dissolution medium was evaluated and compared. The results are shown in Table 13. The results indicate that the release of verapamil hydrochloride from acrylate PSA tablets is not affected by the change in the dissolution medium pH. In comparison, glassy rigid polymer tablet prepared using ethyl cellulose shows that verapamil hydrochloride release is dependent on the pH of the dissolution medium.

| Components | XIII |
|---|---|
| Acrylate PSA | 20 |
| Verapamil hydrochloride | 80 |

Acrylate PSA here is acrylate polymer DURO_TAK 87-202A obtained from Henkel corporation

### Example XIV

### Release of Dilcofenac Sodium from Silicone Tablet Containing PEG3400

In this example, diclofenac sodium tablets were prepared using procedure described in Example III. Effect of addition of solubilizing agent polyethylene glycol on diclofenac sodium drug release from silicone PSA may be evaluated. The effect of addition of calcium phosphate, dibasic on diclofenac sodium was also evaluated. The compositions of silicone PSA tablets are shown in table below.

The conditions for the dissolution study in this example is similar to that explained in Example III, in simulated intestinal fluid about pH 6.8. The rate of diclofenac sodium release from 20 weight percent silicone PSA containing different excipients was evaluated and compared. The results are shown in Table 14. The results indicate that the release of diclofenac sodium is increased by the addition of excipients such as mannitol, calcium phosphate or polyethylene glycol.

| Components | XIVA | XIVB | XIVC | XIVD |
|---|---|---|---|---|
| Silicone PSA | 20 | 20 | 20 | 20 |
| Diclofenac sodium | 80 | 40 | 40 | 40 |
| Mannitol | 0 | 40 | — | — |
| Calcium phosphate, dibasic | — | — | 40 | — |
| PEG 8000 | — | — | — | 40 |

Silicone here is Bio-PSA 7-4202 amine compatible adhesive resin from Dow Corning

### Example XV

### Release of Dexamethasone from Silicone Tablet and Addition of Cyclodextrin Such as 2-Hydropropyl Cyclodextrin

In this example, dexamethasone tablets were prepared using procedure described in Example III. Effect of addition of solubilizing agent hydroxypropyl-beta-cyclodextrin on dexamethasone drug release from silicone PSA may be evaluated. The effect of addition of surfactant on dexamethasone was also evaluated. The compositions of silicone PSA tablets are shown in table below.

The conditions for the dissolution study in this example is similar to that explained in Example III, in simulated intestinal fluid about pH 6.8. The rate of dexamethasone release from 20 weight percent silicone PSA containing different excipients was evaluated and compared. The results are shown in Table 15. The results indicate that the release of dexamethasone can be increased by addition of excipients such as 2-hydroxypropyl-beta-cyclodextrin or surfactant such as Brij98.

| Components | XVA | XVB | XVC |
|---|---|---|---|
| Silicone PSA | 10 | 10 | 10 |
| Dexamethasone | 1.2 | 1.2 | 1.2 |
| Mannitol | 88.8 | 78.8 | 78.8 |
| Hydroxypropyl beta-cyclodextrin | — | 10 | — |
| Brij 98 | — | — | 10 |

Silicone here is Bio-PSA 7-4202 amine compatible adhesive resin from Dow Corning

While various embodiments of the present invention have been described above, it should be understood that they have been presented by ay of example, and not limitation. It will be apparent to persons skilled in the relevant art(s) that various changes in form and detail can be made therein without departing from the scope(s) of the present invention. This, embodiments of this present invention should not be limited by any of the above described exemplary embodiments, but should be defined only in accordance with the following claims and their equivalents.

TABLE 1

| | | APAP | Polymer |
|---|---|---|---|
| Example | Polymer | (% w/w) | (% w/w) |
| 1A | Silicone | 99 | 1 |
| 1B | Silicone | 97.5 | 2.5 |
| 1C | Silicone | 95 | 5 |
| 1D | Silicone | 90 | 10 |
| 1E | Silicone | 85 | 15 |
| 1F | Silicone | 80 | 20 |

TABLE 2
Example

| | 1A | 1B | 1C | 1D | 1E | 1F | 2 |
|---|---|---|---|---|---|---|---|
| Polymer | Silicone | Silicone | Silicone | Silicone | Silicone | Silicone | EC |
| % w/w | 1 | 2.5 | 5 | 10 | 15 | 20 | 20 |
| polymer | | | | | | | |
| % w/w | 99 | 97.5 | 95 | 90 | 85 | 80 | 80 |
| APAP | | | | | | | |
| Tensile | 0.020 | 0.025 | 0.042 | 0.080 | 0.106 | 0.511 | — |
| strength | | | | | | | |
| % | 66.1 | 33.9 | 0.2 | 0.0 | 0.0 | 0.0 | 4.8 |
| Fria- | | | | | | | |
| bility | | | | | | | |
| Contact | 88.2 | 90.2 | 96.8 | 89.5 | 90.1 | 89.1 | 44.1 |
| angle | | | | | | | |
| (Deg) | | | | | | | |

Silicone here is Bio-PSA 7-4202 amine compatible adhesive resin from Dow Corning
EC here is Ethyl cellulose, 48% ethoxy content from International Specialty Polymers Inc

TABLE 3

| | Silicone | | | | | | | |
|---|---|---|---|---|---|---|---|---|
| | adhesive | | | | | | | |
| Example | (w/w) | 0.5 h | 1 h | 2 h | 4 h | 8 h | 12 h | 24 h |
| 1A | 1 | 32.1 | 58.9 | 92.0 | 98.9 | 99.2 | 99.3 | 101.5 |
| 1B | 2.5 | 22.3 | 32.6 | 54.6 | 86.5 | 97.3 | 100.0 | 100.1 |
| 1C | 5 | 31.9 | 31.9 | 46.9 | 66.4 | 93.7 | 99.1 | 102.6 |
| 1D | 10 | 18.0 | 29.9 | 43.8 | 59.1 | 82.7 | 93.9 | 100.7 |
| 1E | 15 | 17.3 | 27.1 | 37.8 | 50.3 | 66.9 | 79.0 | 106.1 |
| 1F | 20 | 11.8 | 17.8 | 24.1 | 36.0 | 50.7 | 63.7 | 82.2 |

Silicone here is Bio-PSA 7-4202 amine compatible adhesive resin from Dow Corning

TABLE 4

| | 0 h | 0.5 h | 1 h | 2 h | 4 h | 8 h | 12 h | 24 h |
|---|---|---|---|---|---|---|---|---|
| pH 1.2 | 0.0 | 13.1 | 20.3 | 28.2 | 39.5 | 52.0 | 60.0 | 74.0 |
| pH 6.8 | 0.0 | 11.8 | 17.8 | 24.1 | 36.0 | 50.7 | 63.7 | 82.2 |

TABLE 5

| Exam- | | | | | | | | | |
|---|---|---|---|---|---|---|---|---|---|
| ple | Force | 0 h | 0.5 h | 1 h | 2 h | 4 h | 8 h | 12 h | 24 h |
| VA | 3.5 kN | 0.0 | 12.9 | 18.9 | 27.4 | 38.9 | 56.1 | 68.9 | 93.6 |
| VB | 6.1 kN | 0.0 | 15.3 | 21.1 | 30.0 | 41.2 | 57.2 | 70.7 | 97.3 |
| VC | 8.7 kN | 0.0 | 11.8 | 17.8 | 24.1 | 36.0 | 50.7 | 63.7 | 82.2 |

TABLE 6

| | 0 h | 0.5 h | 1 h | 2 h | 4 h | 8 h | 12 h | 24 h |
|---|---|---|---|---|---|---|---|---|
| 10% Ethanol | 0.0 | 15.5 | 24.8 | 30.0 | 48.4 | 77.9 | 88.4 | 89.2 |
| FesSIF | 0.0 | 17.4 | 25.3 | 38.5 | 53.6 | 72.7 | 82.0 | 104.8 |
| FasSIF | 0.0 | 15.8 | 23.3 | 43.0 | 52.5 | 69.9 | 77.7 | 103.4 |

TABLE 7

| | 0 h | 0.5 h | 1 h | 2 h | 4 h | 8 h | 12 h | 24 h |
|---|---|---|---|---|---|---|---|---|
| Tramadol HCl | 0.0 | 15.8 | 23.2 | 32.1 | 43.4 | 56.8 | 68.3 | 89.4 |
| APAP | 0.0 | 13.5 | 22.6 | 31.9 | 44.8 | 61.1 | 74.3 | 86.0 |

TABLE 8

| Example | 0 h | 0.5 h | 1 h | 2 h | 4 h | 6 h | 8 h | 10 h | 12 h | 24 h |
|---|---|---|---|---|---|---|---|---|---|---|
| 8A | 0.0 | 15.2 | 23.3 | 36.6 | 54.9 | 66.9 | 74.2 | 80.6 | 85.4 | 102.9 |
| 8B | 0.0 | 8.3 | 11.2 | 15.3 | 20.4 | 24.6 | 27.6 | 30.2 | 32.3 | 43.3 |
| 8C | 0.0 | 25.4 | 38.2 | 58.2 | 84.7 | 96.2 | 98.4 | 99.1 | 99.4 | 102.2 |
| 8D | 0.0 | 18.9 | 27.2 | 39.0 | 56.0 | 66.8 | 75.6 | 82.6 | 90.4 | 100.6 |

TABLE 9

| Example | 0 h | 0.5 h | 1 h | 2 h | 4 h | 6 h | 8 h | 10 h | 12 h | 24 h |
|---|---|---|---|---|---|---|---|---|---|---|
| IXA | 0.0 | 11.8 | 17.8 | 24.1 | 36.0 | — | 50.7 | — | 63.7 | 82.2 |
| IXB | 0.0 | 12.2 | 18.1 | 25.8 | 36.8 | — | 50.7 | — | 60.8 | 84.7 |
| IXC | 0.0 | 14.2 | 16.3 | 20.6 | 29.0 | 43.6 | 56.9 | 83.3 | — | — |
| IXD | 0.0 | 18.0 | 29.9 | 43.8 | 59.1 | 73.1 | 82.7 | 93.9 | 100.7 | — |
| IXE | 0.0 | 18.3 | 28.8 | 41.9 | 58.9 | 69.9 | 81.3 | 92.3 | 101.7 | — |

TABLE 10

| | Mechanical | | | | | | |
|---|---|---|---|---|---|---|---|
| Polymer | stress | 0 h | 1 h | 2 h | 4 h | 8 h | 12 h |
| EC | − | 0 | 56.2 | 79.3 | 120.1 | 174.4 | 242.5 |
| EC | + | 0.0 | 32.5 | 69.5 | 223.1 | 405.5 | 412.4 |
| PSA 7-4102 | − | 0.0 | 78.4 | 98.2 | 187.5 | 240.5 | 244.3 |
| PSA 7-4102 | + | 0.0 | 27.1 | 80.5 | 131.1 | 204.2 | 254.5 |
| PSA 7-4202 | − | 0.0 | 82.1 | 128.3 | 175.1 | 230.4 | 302.4 |
| PSA 7-4102 | + | 0.0 | 58.1 | 85.1 | 126.0 | 187.7 | — |
| PSA 7-4302 | − | 0.0 | 83.4 | 113.0 | 171.6 | 277.4 | 301.2 |
| PSA 7-4302 | + | 0.0 | 33.1 | 56.3 | 85.9 | 139.8 | 167.4 |

PSA 7-4102 here is Bio-PSA 7-4102 amine compatible adhesive resin from Dow Corning
PSA 7-4202 is Bio-PSA 7-4202 amine compatible adhesive resin from Dow Corning
PSA 7-4302 here is Bio-PSA 7-4302 amine compatible adhesive resin from Dow Corning
EC here is Ethyl cellulose, 48% ethoxy content from International Specialty Polymers Inc

TABLE 11

| | Disso- | Poly- | | | | | | | |
|---|---|---|---|---|---|---|---|---|---|
| | lution | mer | | | | | | | |
| | medium | % | | | | | | | |
| Polymer | pH | w/w | 0 h | 0.5 h | 1 h | 2 h | 4 h | 8 h | 12 h |
| EC | 1.2 | 20 | 0.0 | 25.7 | 37.1 | 49.6 | 70.7 | 93.4 | 107.6 |
| EC | 6.8 | 20 | 0.0 | 16.5 | 29.6 | 34.1 | 50.2 | 65.0 | 79.7 |
| Silicone | 1.2 | 20 | 0.0 | 29.7 | 31.7 | 48.9 | 69.0 | 93.6 | 103.8 |
| Silicone | 6.8 | 20 | 0.0 | 26.8 | 33.3 | 45.8 | 67.5 | 90.2 | 98.0 |

TABLE 12

| Example | 0 h | 0.5 h | 1 h | 2 h | 4 h | 8 h | 12 h | 24 h |
|---|---|---|---|---|---|---|---|---|
| XIIA | 0.0 | 5.7 | 6.5 | 8.3 | 11.3 | 17.0 | 23.5 | 30.4 |
| XIIB | 0.0 | 32.6 | 47.7 | 71.7 | 78.5 | 81.2 | 80.3 | 79.6 |
| XIIC | 0.0 | 28.9 | 48.1 | 65.6 | 71.7 | 73.7 | 74.0 | 76.7 |
| XIID | 0.0 | 55.4 | 71.5 | 99.2 | 105.8 | 105.3 | 106.1 | — |

TABLE 13

| | 0 h | 0.5 h | 1 h | 2 h | 4 h | 8 h | 12 h |
|---|---|---|---|---|---|---|---|
| pH 1.2 | 0.0 | 16.2 | 23.0 | 38.3 | 69.7 | 81.0 | 84.9 |
| pH 6.8 | 0.0 | 16.7 | 27.7 | 53.6 | 73.6 | 75.2 | 76.6 |

TABLE 14

| | 0 h | 0.25 h | 0.5 h | 0.75 h | 1 h | 1.5 h | 2 h | 3 h | 4 h | 6 h | 8 h | 12 h | 24 h |
|---|---|---|---|---|---|---|---|---|---|---|---|---|---|
| XIVA | 0.0 | 6.7 | 5.9 | 6.7 | 10.4 | 8.4 | 9.3 | 10.4 | 12.6 | 14.4 | 17.3 | 22.6 | 31.0 |
| XIVB | 0.0 | 7.9 | 10.0 | 12.5 | 14.9 | 18.1 | 21.4 | 25.7 | 30.1 | 36.0 | 41.2 | 48.1 | 60.1 |
| XIVC | 0.0 | 1.8 | 3.4 | 4.5 | 5.3 | 7.2 | 8.9 | 11.5 | 14.4 | 18.1 | 21.9 | 29.2 | 42.4 |
| XIVD | 0.0 | 11.3 | 15.9 | 18.9 | 20.8 | 23.4 | 28.0 | 36.4 | 44.9 | 54.3 | 59.6 | 68.4 | 77.4 |

TABLE 15

| Example | 0 h | 0.5 h | 1 h | 2 h | 4 h | 8 h | 12 h | 24 h |
|---|---|---|---|---|---|---|---|---|
| XVA | 0.0 | 23.6 | 37.6 | 44.0 | 44.3 | 45.7 | 45.6 | 51.1 |
| XVB | 0.0 | 6.5 | 12.4 | 14.1 | 19.8 | 23.3 | 30.0 | 32.3 |
| XVC | 0.0 | 32.6 | 45.6 | 62.7 | 69.8 | 79.4 | 81.0 | 90.8 |

What is claimed is:

1. A solid tablet for controlled release of active medicaments comprising one or more active pharmaceutically agent in an amount sufficient to provide a therapeutic effect when administered and at least a pressures sensitive adhesive.

2. The solid tablet of claim 1 wherein the pressure sensitive adhesive is a silicone pressure sensitive adhesive selected from a silicone/resin copolymer with silicon-bonded hydroxyl radicals, a silicone/resin copolymer with end capped silicon-bonded hydroxyl radicals and mixtures thereof.

3. The solid tablet of claim 2 wherein the pressure sensitive adhesive is an silicone pressure sensitive adhesive selected from a silicone/resin copolymer and has 40 to 70 parts by weight of at least one resin copolymer and 30 to 70 parts by weight of polydiorganosiloxane comprising ARSiO units.

4. A composition according to claim 2, wherein the silicone/resin copolymer is prepared by polycondensation of the silanol endblocked polydialkylsiloxane with a hydroxyl end-blocked silicate resin.

5. The solid tablet of claim 1 wherein the pressure sensitive adhesive is an acrylic pressure sensitive adhesive comprising about 5 to about 100 weight percent of (meth)acrylic acid copolymer cross-linked with a crosslinking agent.

6. The solid tablet of claim 1 wherein the active pharmaceutical agent is selected from the group consisting of analgesics, anti-inflammatory agents, antihelmimthics, anti-arrhythmic agents, anti-bacterial agents, anti-viral agents, anti-coagulants, anti-depressants, anti-diabetics, anti-epileptics, anticancer agent, anti-fungal agents, anti-gout agents, antihypertensive agents, anti-malariale, anti-migrainc agents, anti-muscarinic agents, anti-neoplastic agents, erectile dysfunction improvement agents, immunosuppressants, antiprotozoal agents, anti-thyroid agents, anxiolytic agents, sedatives, hypnotics, neuroleptics, (3-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, antioxidant agent, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, a hormone, a steroid, steroid antagonist, a vitamin, essential fatty acids, non-essential fatty acids, and mixtures thereof.

7. The solid tablet of claim 6 wherein the active pharmaceutical agent is poorly compressible.

8. A solid tablet for controlled release of active medicaments of claim 1 wherein the pressures sensitive adhesive comprises from about 0.1 to about 40 weight %, based on the total weight of the composition for aiding compression and controlling medicament release.

9. A solid tablet for controlled release of active medicaments comprising one or more active pharmaceutically agent in an amount sufficient to provide a therapeutic effect when administered, an excipient and at least a pressures sensitive adhesive.

10. A solid tablet for controlled release of active medicaments of claim 9 wherein the pressures sensitive adhesive comprises from about 0.1 to about 40 weight %, based on the total weight of the composition, the active pharmaceutical agent comprises from about 10 to about 99 weight % based on the total weight of the composition and the excipient comprises from about 5 to about 70 weight % of the total weight of the composition.

11. A method according to claim 1 for controlled and sustained release of medicaments for oral drug delivery delivered from a tablet compromising of silicone pressure sensitive adhesive and at least one active medicament.

12. A method according to claim 1 for controlled and sustained release of medicaments for oral delivery from a tablet along the oral route comprising of a pressure sensitive adhesive, at least an active agent and at least one water soluble excipient comprising of:
(i) an anti-sticking agent, a glidant, a flow promoter, a lubricant, an anticoagulant, an antifoaming agent, an antioxidant, a binder, a buffering agents, a chelating agent, a coagulant, a colorant, a cryoprotectant, a diluent, a filler, a disintegrant, a flavorant, or sweetner, a plasticizer, a preservative and mixtures thereof.

13. A composition of claim 9, wherein a solubilizing agent is added to the oral tablet comprises from about 1 to about 70 weight percent of the total composition.

14. A composition according to claim 9, wherein the solubilizing agent is a complexing agent, surfactant, liposome, PEG derived fatty acids, buffering agent, glycols or sugars.

15. A solid tablet of claim 1, wherein the active pharmaceutical agent is a salt form of the active agent.

16. A solid tablet of claim 1, wherein the active pharmaceutical agent is a weak acid, weak base, non ionizable or mixtures thereof.

17. A method for preparing the oral tablet comprising the steps of:
(i) dissolving the pressure sensitive adhesive in appropriate solvent
(ii) forming a suspension of medicament in the adhesive/solvent solution
(iii) drying the suspension to obtain a solid matrix
(iv) compressing the matrix to form required shape and size of the oral dosage form

18. A method according to claim 17, wherein the solvent used for dissolving the polymer is selected consisting of hydrocarbon solvents, volatile silicones, non-hydrocarbon solvents and mixtures thereof.

19. A method for preparing the solid tablet according to claim 1, wherein the adhesive
(i) is a curable adhesive, cured either by using catalytic reaction, heat, oxidation, light or combinations thereof
(ii) is a cross-linked pressure sensitive adhesive.
